(19) United States
(12) Patent Application Publication
(10) Pub. No.: US 2018/0242826 A1
(43) Pub. Date: Aug. 30, 2018

(54) ENDOSCOPE, IMAGE PICKUP MODULE AND METHOD FOR MANUFACTURING IMAGE PICKUP MODULE

(71) Applicant: OLYMPUS CORPORATION, Tokyo (JP)

(72) Inventor: Takahiro SHIMOHATA, Shiojiri-shi (JP)

(73) Assignee: OLYMPUS CORPORATION, Tokyo (JP)

(21) Appl. No.: 15/961,961

(22) Filed: Apr. 25, 2018

Related U.S. Application Data
(63) Continuation of application No. PCT/JP2015/080098, filed on Oct. 26, 2015.

(51) Int. Cl. A61B 1/04; A61B 1/06; A61B 1/00

(52) U.S. Cl. CPC A61B 1/041; A61B 1/0011; A61B 1/0661

(57) ABSTRACT

An image pickup module includes: an image pickup device; an image pickup optical portion; a frame member in which the image pickup optical portion is arranged; and a wiring board on which the frame member is fixed to a first main surface by a fixing member, and the image pickup device is mounted on a second main surface. A gap exists between a bottom surface of the frame member and the first main surface of the wiring board, and the fixing member is arranged covering the gap.

### CROSS REFERENCE TO RELATED APPLICATION

This application is a continuation application of PCT/JP2015/080098 filed on Oct. 26, 2015, the entire contents of which are incorporated herein by this reference.

### BACKGROUND OF INVENTION

### 1. Field of the Invention

The present invention relates to an endoscope including an image pickup module in which a frame member in which an image pickup optical portion is arranged is fixed to a wiring board where an image pickup device is mounted, the image pickup module in which the frame member in which the image pickup optical portion is arranged is fixed to the wiring board where the image pickup device is mounted, and a method for manufacturing the image pickup module in which the frame member in which the image pickup optical portion is arranged is fixed to the wiring board where the image pickup device is mounted.

### 2. Description of the Related Art

Japanese Patent Application Laid-Open Publication No. 2005-143670 discloses a capsule-type endoscope in which a lens of an image pickup optical portion is fixed in a manner of being in close contact with a light receiving surface of an image pickup device.

Japanese Patent Application Laid-Open Publication No. 2004-248753 discloses a capsule-type medical apparatus electrically connecting an MID (molded interconnect device) with a wiring board and the like by solder bond.

### SUMMARY OF THE INVENTION

An endoscope of an embodiment of the present invention includes an image pickup module provided with: an image pickup device; an image pickup optical portion configured to collect light to the image pickup device; a frame member which is a molded interconnect device with non-conductive resin as basic material, the frame member including wiring, the image pickup optical portion being arranged in the frame member; a light emitting device mounted on the molded interconnect device; and a wiring board including a first main surface and a second main surface facing the first main surface, the frame member being fixed to the first main surface by a fixing member, the image pickup device being mounted on the first main surface or the second main surface. A gap exists between a bottom surface of the frame member and the first main surface of the wiring board; the fixing member is arranged covering the gap; and the wiring is connected to the wiring board via a conductive member made of conductive paste or solder, the conductive member being arranged covering the gap.

An image pickup module of another embodiment is an image pickup module provided with: an image pickup device; an image pickup optical portion configured to collect light to the image pickup device; a frame member in which the image pickup optical portion is arranged; and a wiring board including a first main surface and a second main surface facing the first main surface, the frame member being fixed to the first main surface by a fixing member, the image pickup device being mounted on the first main surface or the second main surface. A gap exists between a bottom surface of the frame member and the first main surface of the wiring board.

Further, a method for manufacturing an image pickup module of another embodiment includes steps of: fabricating an image pickup device, an image pickup optical portion arranged in a frame member, and a wiring board including a first main surface and a second main surface; mounting the image pickup device on the wiring board; attaching a holding jig to the frame member or the wiring board; disposing the frame member on the first main surface of the wiring board and performing alignment by moving the holding jig in a state where a gap exists between a bottom surface of the frame member and the first main surface of the wiring board; performing solidification treatment for a fixing member in a liquefied state to fix the frame member to the first main surface; and removing the holding jig from the frame member or the wiring board.

### BRIEF DESCRIPTION OF THE DRAWINGS

FIG. 1 is a cross-sectional view of an image pickup module of a first embodiment;

FIG. 2 is an exploded perspective view of the image pickup module of the first embodiment;

FIG. 3 is a flowchart for illustrating a method for manufacturing the image pickup module of the first embodiment;

FIG. 4 is a cross-sectional view for illustrating the method for manufacturing the image pickup module of the first embodiment;

FIG. 5 is a cross-sectional view of an image pickup module of a modification of the first embodiment;

FIG. 6 is a cross-sectional view of an image pickup module of a second embodiment;

FIG. 7 is a cross-sectional view of an image pickup module of a third embodiment;

FIG. 8 is an exploded perspective view of the image pickup module of the third embodiment;

FIG. 9 is a cross-sectional view of an image pickup module of a first modification of the third embodiment;

FIG. 10 is a cross-sectional view of an image pickup module of a second modification of the third embodiment;

FIG. 11 is an exploded perspective view of an image pickup module of a fourth embodiment; and

FIG. 12 is a cross-sectional view of an endoscope of a fifth embodiment.

### DETAILED DESCRIPTION OF THE PREFERRED EMBODIMENTS

### First Embodiment

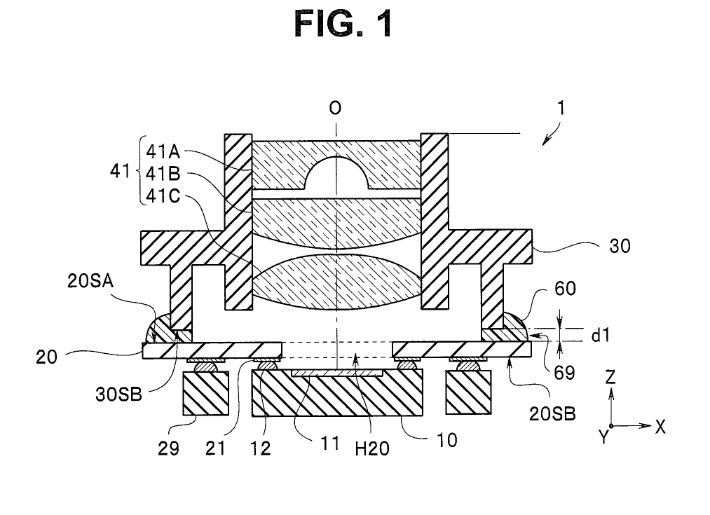

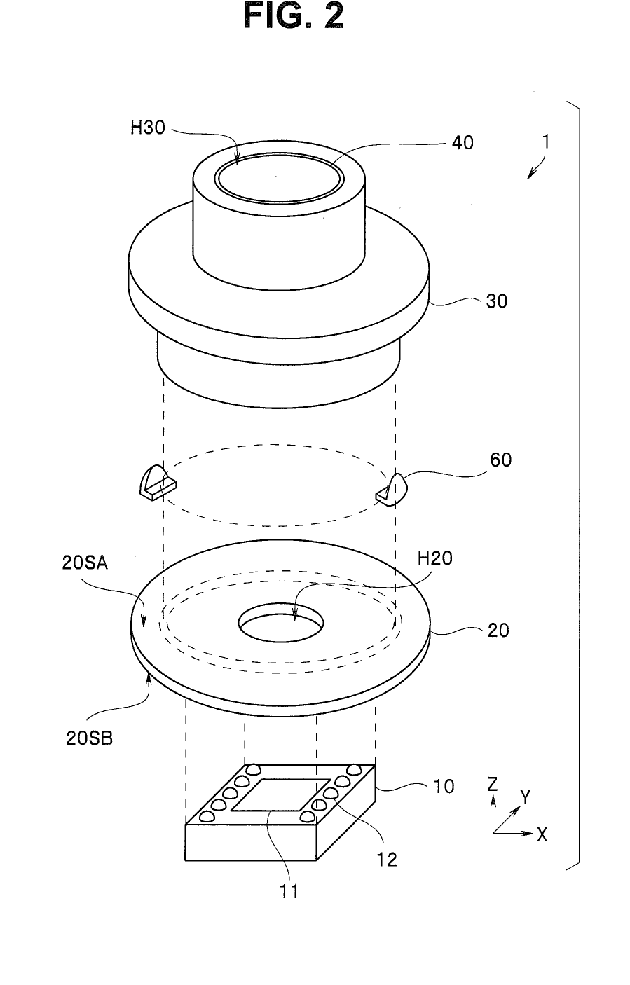

As shown in FIGS. 1 and 2, an image pickup module 1 of an embodiment of the present invention is provided with an image pickup device 10, a wiring board 20, a frame member 30 and an image pickup optical portion 40.

Note that, in description below, it should be noted that drawings based on each embodiment are schematic, and a relationship between thickness and width of each portion, a thickness ratio among the portions, and the like are different from actual ones; and in some cases, portions with a different mutual dimension relationship or ratio may be included among the drawings. Further, in some cases, some of components may be not shown.

On a light receiving surface of the image pickup device 10 configured with a semiconductor such as silicon, a light receiving portion 11 configured with a CCD or CMOS image pickup circuit or the like is formed. On an outer peripheral portion of the light receiving portion 11, a plurality of external connection electrodes 12 which are electrically connected with the light receiving portion 11 are arranged.

The image pickup optical portion 40 in a cylindrical shape which includes a plurality of lenses 41 (41A to 41C) collects light to the image pickup device 10.

In a through hole H30 of the frame member 30, which is made of metal, resin or ceramics, the image pickup optical portion 40 is inserted and fixed. The frame member 30 has a bottom surface 30SB disposed facing a first main surface 20SA of the wiring board 20.

Note that, though the frame member 30 is in a shape configured with a combination of a plurality of hollow cylinders having different diameters, the frame member 30 may be a simple hollow cylinder the outer surface of which has a same diameter or may be a hollow polygonal prism or the like. Further, the frame member 30 may not be one body but may be configured with a combination of a plurality of cylinders and the like. Furthermore, cross-sectional shapes of the image pickup optical portion 40 and a through hole H20 may be, for example, polygons.

To the first main surface 20SA of the wiring board 20 which is substantially donut-shaped, the frame member 30 is fixed by fixing members 60 made of UV curable resin. On a second main surface 20SB facing the first main surface 20SA of the wiring board 20, the image pickup device 10 is mounted. Light from the image pickup optical portion 40 enters the image pickup device 10 via the through hole H20 in the middle of the wiring board 20. On the second main surface 20SB of the wiring board 20, electronic parts 29 such as a chip capacitor are mounted.

Note that, though not shown, the wiring board 20 is connected to a processing circuit configured to process an image pickup signal of the image pickup device 10, a drive circuit configured to output a drive signal to the image pickup device 10 and the like via a flexible wiring board, an MID, a cable and the like.

In an image pickup module 1, a gap 69 exists between the bottom surface 30SB of the frame member 30 and the first main surface 20SA of the wiring board 20. Therefore, the fixing members 60 fixing the frame member 30 and the wiring board 20 are arranged covering the gap 69 with a length d1. That is, the frame member 30 and the wiring board 20 are not directly in contact with each other at any part. The frame member 30 and the wiring board 20 are in so-called aerial connection with each other via the fixing members 60.

In a small-size image pickup module, especially in an image pickup module for endoscope, positioning (optical axis alignment) between an image pickup optical portion and an image pickup device is not easy.

In the image pickup module 1, positioning (light axis alignment) between the image pickup optical portion 40 arranged in the frame member 30 and the image pickup device 10 mounted on the wiring board 20 is performed in a state where the frame member 30 and the wiring board 20 are not in contact with each other in order to realize fine adjustment as described later. The frame member 30 and the wiring board 20 are not in contact with each other even in a state of being positioned at optimal positions. The frame member 30 and the wiring board 20 are fixed in a manner of covering a gap via the fixing members 60 arranged in a liquid state and solidification-treated. Therefore, optical axis alignment for the image pickup module 1 is accurately performed.

### <Method for Manufacturing Image Pickup Module>

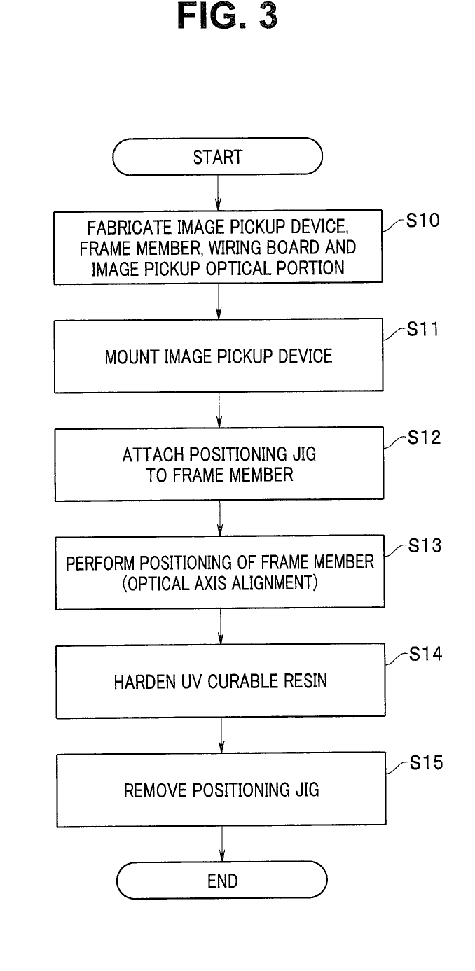

Next, a method for manufacturing the image pickup module 1 will be described along a flowchart shown in FIG. 3.

### <Step S10>

The image pickup device 10, the image pickup optical portion 40 arranged in the frame member 30, and the wiring board 20 are fabricated.

The image pickup device 10 is fabricated, for example, by cutting a silicon wafer on which a plurality of light receiving portions 11 or the like are formed, using a well-known semiconductor technique. The image pickup optical portion 40 is configured with the plurality of lenses 41 made of transparent resin, glass or the like. Spacers, an aperture and the like are arranged among the plurality of lenses 41 though it is not shown. Further, the image pickup optical portion 40 may be attached to a cylindrical lens frame in advance before being arranged in the frame member 30.

As shown in FIG. 2, the image pickup optical portion 40 is inserted into the through hole H30 of the frame member 30 and fixed. An inner diameter of the through hole H30 is set slightly larger than an outer diameter of the image pickup optical portion 40. Therefore, relative positions of the frame member 30 and the image pickup optical portion 40 vary due to manufacturing variation.

The donut-shaped wiring board 20 has a plurality of electrode pads 21 on which the image pickup device 10 or the electronic parts 29 are surface-mounted, on the second main surface 20SB.

### <Step S11>

The image pickup device 10 is mounted on the electrode pads 21 of the wiring board 20. For example, a reflow process is performed in a state where solder bumps, which are the external connection electrodes 12 of the image pickup device 10, are in contact with the electrode pads 21 of the wiring board 20. At this time, the electronic parts 29 are also mounted in a method similar to the method for the image pickup device 10.

Note that, in a case where gold stud bumps are used as the external connection electrodes 12, the mounting on the electrode pads 21 of the wiring board 20 may be performed by flip chip bonding via NCP (non-conductive paste).

Relative positions of the wiring board 20 and the image pickup device 10 vary due to manufacturing variation caused by mounting conditions and the like.

### <Step S12>

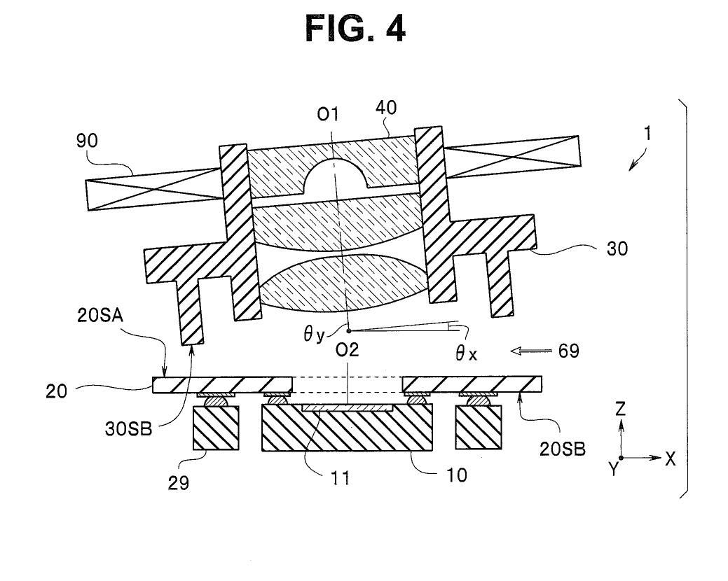

As shown in FIG. 4, a holding jig 90 for aligning a position and an angle of an optical axis relative to the image pickup device 10 and a distance to the image pickup device 10 is attached to the frame member 30. The attachment may be performed by mechanical sandwiching or by adsorption or the like. On the other hand, the wiring board 20 is fixed to a positioning stage. The holding jig 90 is connected to a movable portion for which fine adjustment of an in-plane direction (an XY direction), a vertical direction (a Z direction) and an inclination (xθ, yθ) can be performed based on the positioning stage.

Of course, on the contrary, the frame member 30 may be fixed to the positioning stage, and the holding jig 90 may be attached to the wiring board 20.

### <Step S13>

As shown in FIG. 4, alignment is performed in a state where the frame member 30 is disposed on the first main surface 20SA of the wiring board 20, and the gap 69 exists between the bottom surface 30SB of the frame member 30 and the first main surface 20SA of the wiring board 20.

In a case of an extremely small-sized image pickup module, it is not easy to perform accurate positioning, especially, accurate alignment an optical axis O1 of the frame member 30 (the image pickup optical portion 40) with an optical axis O2 of the image pickup device 10.

In the image pickup module 1, however, design work is performed so that an optimal focus position is obtained in the state where the gap 69 exists without the frame member 30 and the wiring board 20 being in contact with each other. For example, design work is performed so that the optimal focus position is obtained when the length d1 of the gap 69 between the frame member 30 and the wiring board 20 is between 20 μm and 200 μm, including 20 μm and 200 μm.

As already described, the relative position of the image pickup optical portion 40 arranged in the frame member 30 relative to the bottom surface 30SB varies due to manufacturing variation. Similarly, the relative position of the image pickup device 10 mounted on the wiring board 20 relative to the first main surface 20SA varies due to manufacturing variation.

Therefore, if design work is performed so that the optimal focus position is obtained in a state where the bottom surface 30SB of the frame member 30 and the first main surface 20SA of the wiring board 20 are in contact with each other or are fitted together, accuracy of alignment between the image pickup optical portion 40 and the image pickup device 10 decreases due to manufacturing variation.

In comparison, in the image pickup module 1, it is possible to perform positioning between the image pickup optical portion 40 and the image pickup device 10 without being influenced by manufacturing variation.

For example, if the length d1 of the gap 69 at which the optimal focus position is obtained is set to a value between 20 μm and 200 μm, including 20 μm and 200 μm, it is possible to easily perform accurate alignment between the image pickup optical portion 40 and the image pickup device 10 with an accuracy of ±10 μm.

### <Step S14>

In the state where positioning is performed so that the optimal focus position is obtained, the gap 69 exists between the frame member 30 and the wiring board 20.

Then, by performing solidification treatment for the fixing members 60 in a liquefied state, which are arranged in a manner of covering the gap 69, the frame member 30 is fixed to the first main surface 20SA of the wiring board 20.

The liquefied fixing members 60 may be arranged at least either on the frame member 30 or on the wiring board 20 before positioning is performed or may be arranged in a manner of covering the gap 69 after positioning is performed.

As for the fixing members 60, UV irradiation treatment is adopted as the solidification treatment if UV curable resin is used for the fixing members 60, and heat treatment is used as the solidification treatment if thermosetting resin is used for the fixing members 60. Solder can be used as the fixing members 60. For example, if solder disposed in a manner of covering the gap 69 is liquefied by being heated and melted by a laser or the like, the solidification treatment is natural cooling.

If one fixing member 60 covers the gap 69, the frame member 30 is fixed to the wiring board 20. It is, however, desirable that the frame member 30 is fixed by two fixing members 60 so that at least two positions of the gap 69 are covered. Further, the fixing members 60 may be arranged over the entire circumference of the gap 69 in a donut shape.

### <Step S15>

Then, when the holding jig 90 is removed from the frame member 30 after the frame member 30 is fixed, the image pickup module 1 is completed.

As described above, according to the method for manufacturing the image pickup module 1 of the present embodiment, it is possible to easily perform accurate alignment.

### Modification of First Embodiment

As already described, in the image pickup module 1, the through hole H20 exists in the wiring board 20; the image pickup device 10 is mounted on the second main surface 20SB of the wiring board 20; and light collected by the image pickup optical portion 40 enters the image pickup device 10 via the through hole H20.

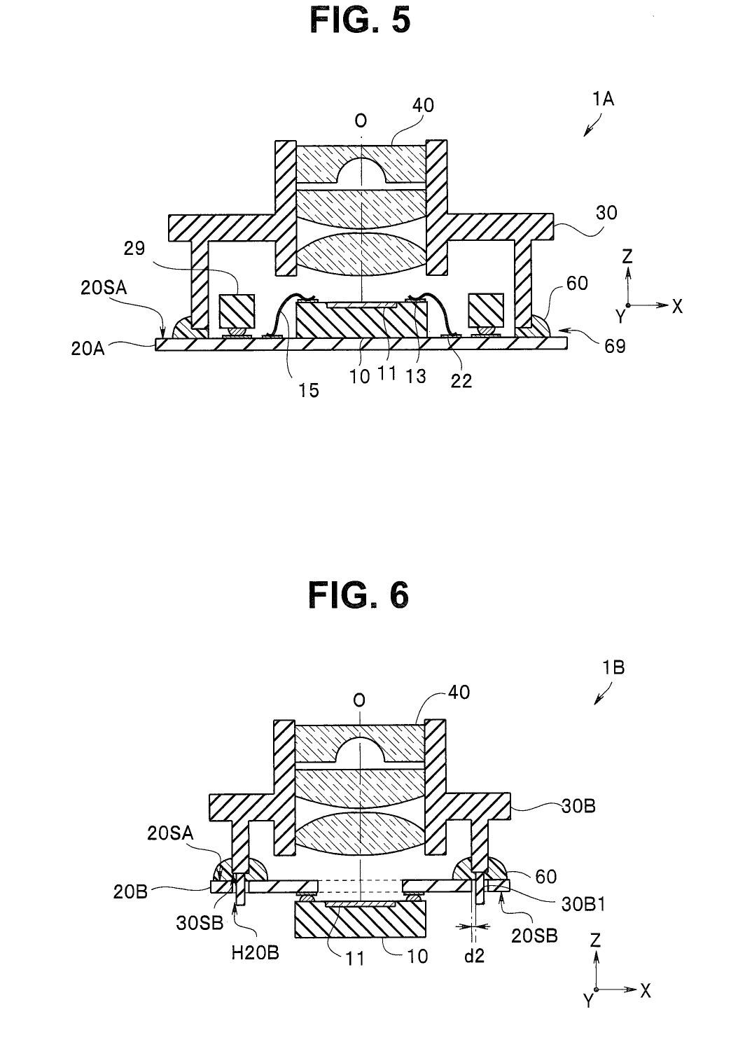

In comparison, in an image pickup module 1A of a modification, the image pickup device 10 is disposed on the first main surface 20SA of a wiring board 20A as shown in FIG. 5. External connection electrodes 13 of the image pickup device 10 are connected to electrode pads 22 on the first main surface 20SA of the wiring board 20A via wire bonding wiring 15.

Further, the electronic parts 29 are also mounted on the first main surface 20SA of the wiring board 20A. Note that the electrode pads 22 connected to the image pickup device 10 may be arranged on the second main surface 20SB of the wiring board 20A via through wiring or the like, and the electronic parts 29 may be mounted only on the second main surface 20SB.

The image pickup module 1A has the effects of the image pickup module 1, and, furthermore, fabrication of the wiring board 20A is easy. Furthermore, since the image pickup device 10 is surrounded by the frame member 30, possibility that natural light enters the light receiving portion 11 does not exist.

### Second Embodiment

Next, an image pickup module 1B of a second embodiment will be described. Since the image pickup module 1B resembles the image pickup module 1, same components will be given a same reference numeral, and description will be omitted.

As shown in FIG. 6, in the image pickup module 1B, projection portions (leg portions) 30B1 exist for simplified positioning in the in-plane direction (the XY direction) on the bottom surface 30SB of a frame member 30B, and holding holes H20B corresponding to the projection portions 30B1 exist on a wiring board 20B.

For example, the projection portions 30B1 in a cylindrical shape are inserted in the holding holes H20B with a circular cross-section. An external diameter of the projection portions 30B1 is smaller than an inner diameter of the holding holes H20B by d2. Therefore, the projection portions 30B1 are loosely fitted in the holding holes H20B. The gap d2 is set in consideration of manufacturing variation.

When the projection portions 30B1 are cylinders, positioning in the in-plane direction (the XY direction) between the frame member 30B and the wiring board 20B is easy if at least two projection portions 30B1 exist. When the projection portions 30B1 are polygonal prisms, positioning in the in-plane direction (the XY direction) is easy even in the case of one projection portion 30B1.

That is, the image pickup module 1B has the effects of the image pickup module 1, and, furthermore, positioning is easy.

Note that, in the image pickup module 1B also, the image pickup device 10 may be disposed on the first main surface 20SA of the wiring board 20B as in the image pickup module 1A though it is not shown.

### Third Embodiment

Next, an image pickup module 1C of a third embodiment will be described. Since the image pickup module 1C resembles the image pickup module 1 and the like, same components will be given a same reference numeral, and description will be omitted.

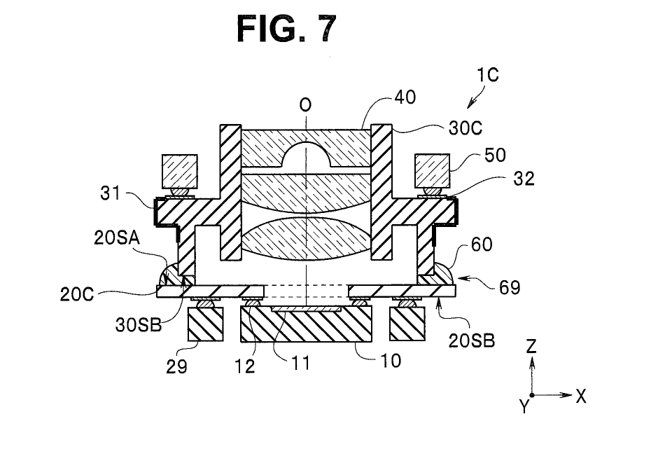

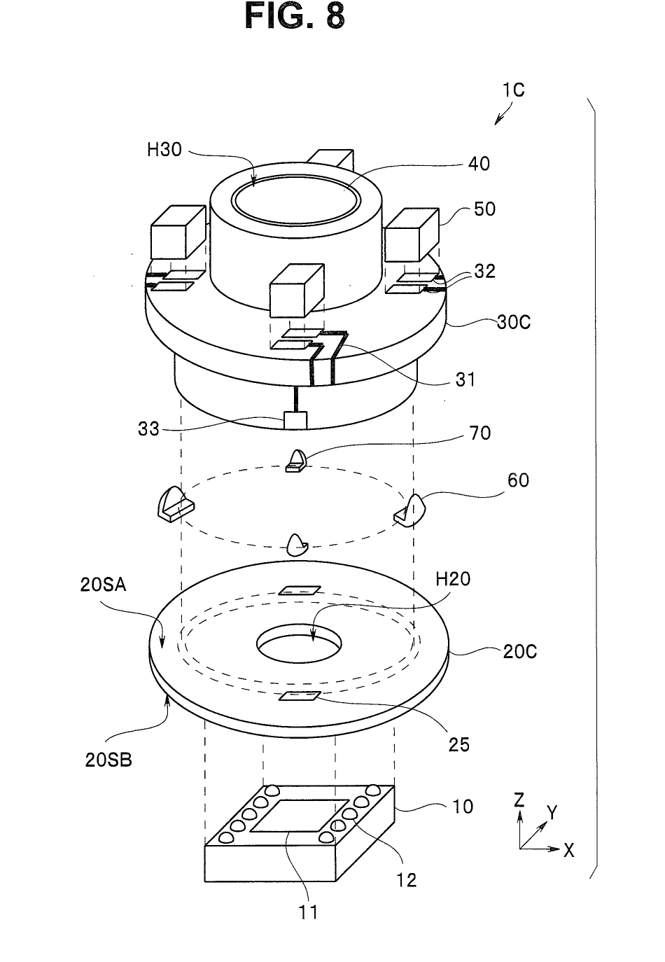

As shown in FIGS. 7 and 8, in the image pickup module 1C, a frame member 30C is a molded interconnect device (an MID) the basic material of which is non-conductive resin and which has wiring 31. The image pickup module 1C is provided with light-emitting devices 50 mounted on the electrode pads 32 of the wiring 31 of the frame member 30C. That is, in the image pickup module 1C, the image pickup optical portion 40 on which the light-emitting devices 50 are integrally molded is mounted on the frame member 30C.

Electrode pads 33 of the wiring 31 are connected to electrode pads 25 of a wiring board 20C via conductive members 70 made of, for example, conductive paste.

Note that, as shown in FIG. 8, though the image pickup module 1C is provided with four light-emitting devices 50 each of which is mounted on two electrode pads 32, the number of electrode pads 33 is two due to ingenious devices for the wiring 31 from the electrode pads 32 to the electrode pads 33. Of course, more electrode pads 33, more conductive members 70 and more electrode pads 25 may be connected to the electrode pads 32.

The basic material of the frame member 30C is non-conductive resin, especially, engineering plastic which can be molded. The basic material is made of, for example, PA (polyamide), PC (polycarbonate), LCP (liquid crystal polymer), PEEK (polyether ether ketone), nylon, PPA (polyphthalamide), ABS (acrylic nitrile/butadiene/styrene resin), material obtained by mixing inorganic filler with the above, and the like.

Conductive patterns of the wiring 31, the electrode pads 32 and 33 and the like of the frame member 30 are fabricated by a laser removal method, a pattern plating method or the like. In the laser removal method, after a conductive film is formed on a whole surface of a molded body by a plating method or the like, unnecessary portions are removed by laser irradiation. In the pattern plating method, a mask pattern is arranged on a surface of a molded body, and a plating film is formed on areas which are not covered with the mask. Further, by patterning an electroless plating catalyst layer, a plating film may be formed only on areas having the catalyst layer.

A conductive pattern is made of low electrical resistance material such as gold and copper, and it may include a single-layer or multi-layer base film made of titanium, chrome, nickel or the like for improvement of close adhesion. Further, a plurality of pieces of wiring 31 may three-dimensionally cross, with insulating layers arranged among the plurality of pieces of wiring 31.

The light-emitting devices 50 configured with LEDs or the like are mounted on an upper part of the frame member 30C in a manner of emitting light in a direction parallel to an optical axis direction 0 of the image pickup optical portion 40. A surface on which the light-emitting devices 50 are arranged is orthogonal to the optical axis O2 of the image pickup device 10. Therefore, optical performance of the light-emitting devices 50 is improved, and flare and shading are suppressed.

The gap 69 exists between the bottom surface 30SB of the frame member 30C and the first main surface 20SA of the wiring board 20C. Therefore, the conductive members 70 connecting the electrode pads 33 of the wiring board 20C and the electrode pads 25 of the wiring board 20C are arranged to cover the gap 69.

When the conductive paste, which includes low electrical resistance metal particles, a binder and a solvent, dries and solidifies, the conductive members 70 is obtained. The metal particles are made of at least one kind of metal, for example, selected from among nickel, gold, silver, copper, aluminum, iron and the like. As the binder, for example, ethyl cellulose resin, nitrocellulose resin, acrylic resin, alkyd resin, or a combination of the above is used. As the solvent, α-Terpineol, xylene, toluene, diethylene glycol monobutyl ether, diethylene glycol monobutyl ether acetate, diethylene glycol monoethyl ether, diethylene glycol monoethyl ether acetate, and the like are used individually or in combination.

Note that, though a percentage of an amount of the conductive powder in the conductive paste is not especially limited, 70 to 90 weight % is generally preferable, and especially 75 to 85 weight % is preferable.

The liquefied (gel-like) conductive paste becomes solid when the solvent evaporates at a room temperature or a low temperature of 100° C. or below and becomes the low-resistance conductive members 70.

As the conductive members 70, solder or the like may be used.

For example, after the frame member 30C is fixed to the wiring board 20C with the fixing members 60, the frame member 30C and the wiring board 20C are electrically connected by the conductive members 70. Note that, at the time of performing the solidification treatment for fixing the frame member 30C to the wiring board 20C with the fixing members 60, the solvent of the conductive paste may be simultaneously evaporated to obtain the conductive members 70.

The image pickup module 1C has the effects of the image pickup module 1, and, furthermore, electrical connection between the light-emitting devices 50 and the wiring board 20C is easy. Further, by performing power supply to the light-emitting devices 50 by connection of the image pickup device 10 and via the electrode pads 32 of the frame member 30, it is possible to detect a connection error of the image pickup device 10 based on whether the light-emitting devices 50 emit light or not.

Note that, in the image pickup module 1C also, by disposing the image pickup device 10 on the first main surface 20SA of the wiring board 20C, same effects as the image pickup module 1A can be obtained.

### Modification of Third Embodiment

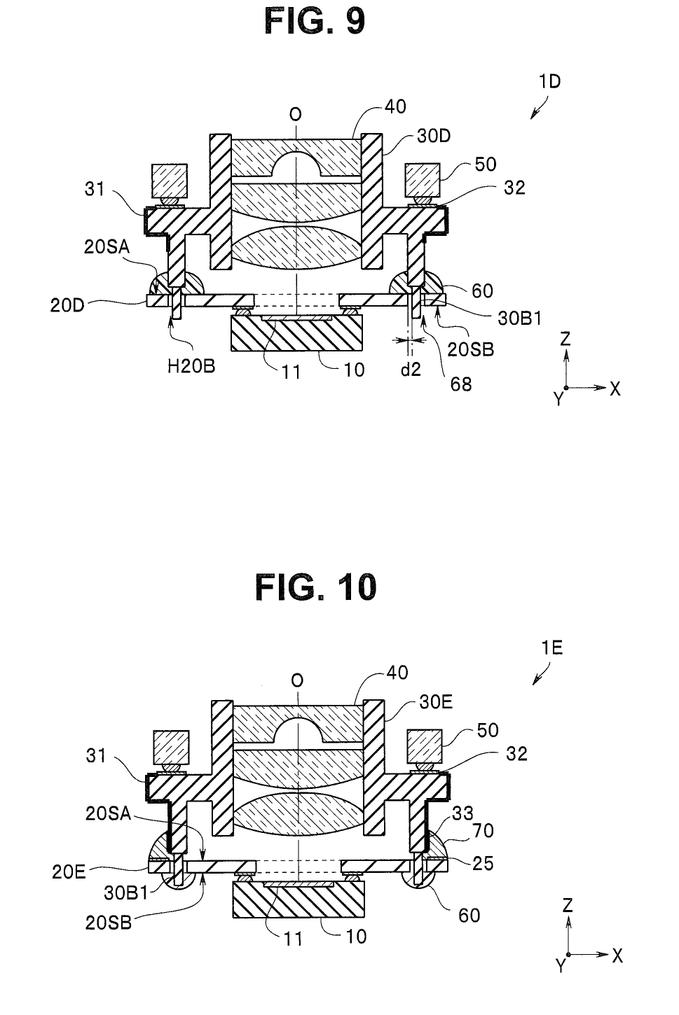

In an image pickup module 1D of a first modification of the third embodiment shown in FIG. 9, the projection portions 30B1 exist on a bottom surface of a frame member 30D, the holding holes H20B exist in a wiring board 20D, and the projection portions 30B1 are loosely fitted in the holding holes H20B, similarly to the image pickup module 1B of the second embodiment.

The image pickup module 1D has the effects of the image pickup modules 1B and 1C.

An image pickup module 1E of a second modification of the third embodiment shown in FIG. 10 resembles the image pickup module 1D. In the image pickup module 1E, however, the fixing members 60 and the conductive members 70 are arranged on different main surfaces of a wiring board 20E.

That is, the fixing members 60 are arranged on the second main surface 20SB of the wiring board 20E, and the conductive members 70 are arranged on the first main surface 20SA. Of course, the fixing members 60 may be arranged on the first main surface 20SA of the wiring board 20E, and the conductive members 70 may be arranged on the second main surface 20SB. In other words, the wiring 31 may be arranged on the projection portions 30B1, and the wiring 31 may be connected to conductors on the second main surface 20SB of the wiring board 20E.

If the number of conductive members 70 and fixing members 60 is large, arrangement of the conductive members 70 and fixing members 60 may not be easy. The image pickup module 1E has the effects of the image pickup module 1D. Furthermore, even if the number of conductive members 70 and fixing members 60 is large, arrangement of the conductive members 70 and the fixing members 60 is easy. Especially, in the image pickup module 1E, possibility that adjoining conductive members 70 are short-circuited does not exist.

### Fourth Embodiment

Next, an image pickup module 1F of a fourth embodiment will be described. Since the image pickup module 1F resembles the image pickup module 1C and the like, same components will be given a same reference numeral, and description will be omitted.

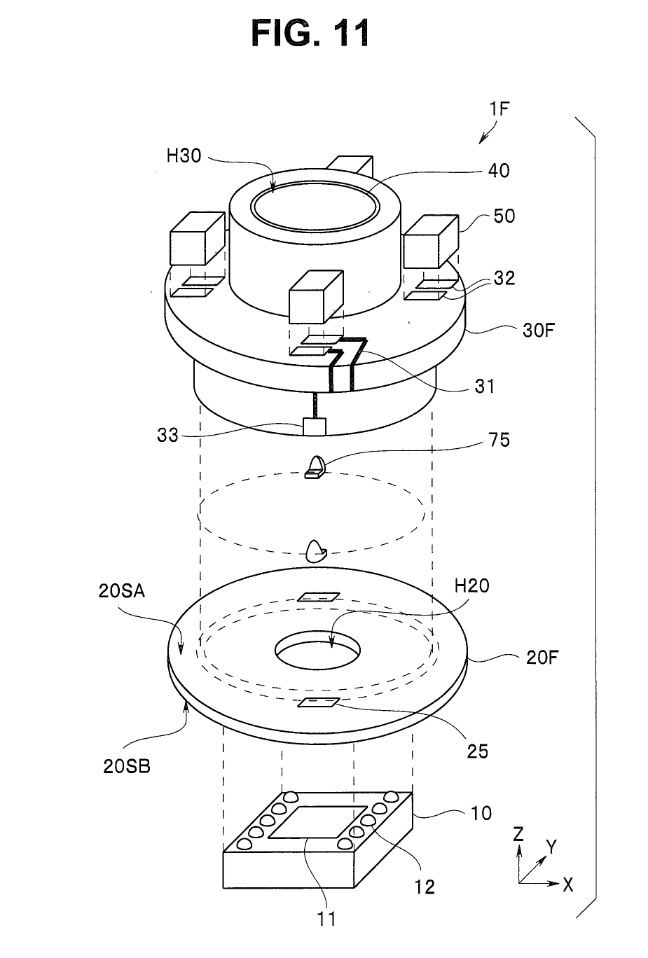

As shown in FIG. 11, in the image pickup module 1F, conductive members 75 also serve as fixing members. In other words, in the image pickup module 1F, fixing members only for fixing a frame member 30F and a wiring board 20F are unnecessary.

For example, if the conductive members 75 are formed by solder, the conductive members 75 have necessary and sufficient characteristics as fixing members and have a function as fixing members. In the image pickup module 1F, the conductive members 75 formed by solder are also fixing members.

The image pickup module 1F has the effects of the image pickup module 1C, and, furthermore, configuration is simple, and manufacture is easy. Note that it goes without saying that, in the image pickup module 1F also, similar effects can be obtained by configuring the image pickup module 1F like the image pickup module 1A though it is not shown.

### Fifth Embodiment

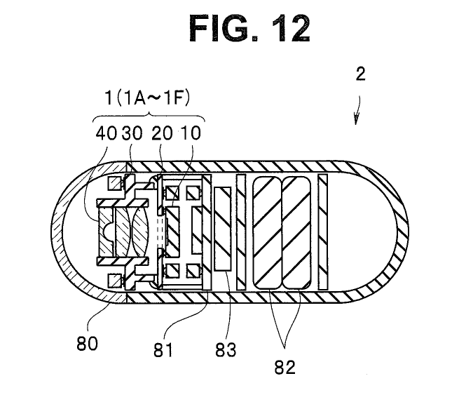

Next, an endoscope 2 of a fifth embodiment of the present invention will be described. As shown in FIG. 12, the endoscope 2 is a capsule-type endoscope. The endoscope 2 includes the image pickup module 1 sealed inside a case 80. The image pickup module 1 is provided with the image pickup device 10, the wiring board 20, the frame member 30 and the image pickup optical portion 40.

Furthermore, a signal transmitting portion 81 configured to wirelessly transmit an image signal outputted by the image pickup module 1, a battery 82 which is a power supply source, a control portion 83 configured to perform overall control, and the like are included inside the case 80.

The case 80 is in an elongated capsule shape, and an end portion on the image pickup module 1 side is in a dome shape and configured with transparent material. A cylindrical portion in the middle and an opposite-side end portion of the dome shape are configured with light-shielding material. Note that, as the endoscope of the embodiment, a so-called binocular capsule type endoscope, which includes the image pickup module 1 on each of both end portions, and both end portions of which are configured with transparent material, may be adopted.

Note that, if any one of the image pickup modules 1A to 1F is included, the endoscope of the embodiment has the effects of each of the image pickup modules.

For example, the endoscope of the present invention is an endoscope including an image pickup module provided with: an image pickup device; an image pickup optical portion configured to collect light to the image pickup device; a frame member which is a molded interconnect device with non-conductive resin as basic material, the frame member including wiring, the image pickup optical portion being arranged in the frame member; a light emitting device mounted on the wiring of the frame member; and a wiring board including a first main surface and a second main surface, the frame member being fixed to the first main surface by fixing members, the image pickup device being mounted on the first main surface or the second main surface. A gap exists between a bottom surface of the frame member and the first main surface of the wiring board; the fixing members are arranged covering the gap; and the wiring is connected to the wiring board via conductive members arranged covering the gap.

Furthermore, in the above endoscope, for example, by using conductive members made of solder, the function of the fixing members can be given to the conductive members.

Furthermore, if the endoscope of the embodiment is provided with any one of the image pickup modules 1, and 1A to 1F for which light axis alignment is accurately performed, the endoscope has the effects of each of the image pickup modules even if the endoscope is a flexible endoscope or a rigid endoscope having an elongated insertion portion.

The present invention is not limited to the embodiments, modifications and the like described above, and various changes, combinations and applications are possible within a range not departing from the spirit of the invention.

What is claimed is:

1. An endoscope comprising:
an image pickup device;
an image pickup optical portion configured to collect light to the image pickup device;
a frame member which is a molded interconnect device with non-conductive resin as basic material, the frame member including wiring, the image pickup optical portion being arranged in the frame member;
a light emitting device mounted on the molded interconnect device; and
a wiring board including a first main surface and a second main surface facing the first main surface, the frame member being fixed to the first main surface by a fixing member, the image pickup device being mounted on the first main surface or the second main surface; wherein
a gap exists between a bottom surface of the frame member and the first main surface of the wiring board;
the fixing member is arranged covering the gap; and
the wiring is connected to the wiring board via a conductive member made of conductive paste or solder, the conductive member being arranged covering the gap.

2. The endoscope according to claim 1, wherein the conductive member made of solder is also the fixing member.

3. An image pickup module comprising:
an image pickup device;
an image pickup optical portion configured to collect light to the image pickup device;
a frame member in which the image pickup optical portion is arranged; and
a wiring board including a first main surface and a second main surface facing the first main surface, the frame member being fixed to the first main surface by a fixing member, the image pickup device being mounted on the first main surface or the second main surface; wherein
a gap exists between a bottom surface of the frame member and the first main surface of the wiring board.

4. The image pickup module according to claim 3, wherein the fixing member is arranged covering the gap.

5. The image pickup module according to claim 3; wherein
a projection portion exists on the bottom surface of the frame member;
a holding hole exists in the wiring board; and
the projection portion is loosely fitted in the holding hole.

6. The image pickup module according to claim 5, wherein the fixing member arranged on the second main surface fixes the projection portion protruding to the second main surface.

7. The image pickup module according to claim 3, wherein the fixing member is made of UV curable resin.

8. The image pickup module according to claim 3, wherein
the frame member is a molded interconnect device with non-conductive resin as basic material, the frame member including wiring;
the image pickup module further comprises a light emitting device mounted on the wiring of the frame member; and
the wiring is connected to the wiring board via a conductive member made of conductive paste or solder.

9. The image pickup module according to claim 8, wherein one of the fixing member and the conductive member is disposed on the first main surface, and another of the fixing member and the conductive member is disposed on the second main surface.

10. The image pickup module according to claim 3; wherein
the frame member is a molded interconnect device with non-conductive resin as basic material, the frame member including wiring;
the image pickup module further comprises a light emitting device mounted on the wiring of the frame member;
the wiring is connected to the wiring board via a conductive member made of solder; and
the wiring is also the fixing member.

11. The image pickup module according to claim 3; wherein
a through hole exists in the wiring board;
the image pickup device is mounted on the second main surface of the wiring board; and
the light collected by the image pickup optical portion enters the image pickup device via the through hole.

12. The image pickup module according to claim 3, wherein the image pickup device is mounted on the first main surface of the wiring board.

13. A method for manufacturing an image pickup module, the method comprising steps of:
fabricating an image pickup device, an image pickup optical portion arranged in a frame member, and a wiring board including a first main surface and a second main surface;
mounting the image pickup device on the wiring board;
attaching a holding jig to the frame member or the wiring board;
disposing the frame member on the first main surface of the wiring board and performing alignment by moving the holding jig in a state where a gap exists between a bottom surface of the frame member and the first main surface of the wiring board;
performing solidification treatment for a fixing member in a liquefied state to fix the frame member to the first main surface; and
removing the holding jig from the frame member or the wiring board.
